(19) United States
(12) Patent Application Publication
(10) Pub. No.: US 2003/0208130 A1
(43) Pub. Date: Nov. 6, 2003

(54) Chest piece for stethoscopes, and methods of utilizing stethoscopes for monitoring the physiological conditions of a patient

(76) Inventors: Dorith Yotam, Ramat Gan (IL); Tommy Schonfeld, Petah Tikva (IL)

Correspondence Address: G E Ehrlich, Suite 207, 2001 Jefferson Davis Highway, Arlington, VA, 22202, US

(21) Appl. No.: 10/297,932

(22) PCT Filed: Jun. 21, 2001
(86) PCT No.: PCT/IL01/00566

(30) Foreign Application Priority Data
Jun. 22, 2000 (IL) 136943
Jun. 27, 2000 (IL) 137047

(51) Int. Cl. A61B 5/02

(52) U.S. Cl. 600/528

(57) ABSTRACT

A stethoscope chest piece includes a housing having a rigid contact surface to be pressed against a patient's chest for picking-up sounds therefrom; a mass yieldingly mounted with respect to the housing and the contact surface; and an electrical transducer in the housing and cooperable with the mass and the contact surface to generate an electrical signal corresponding to the displacement of the contact surface relative to the mass. Also described are a stethoscope apparatus including processing circuitry for reducing noise, a loudspeaker for converting the output of the chest piece to sound, various features making the apparatus attractive to children, and a method of using the apparatus for monitoring a physiological condition of a patient.

### FIELD AND BACKGROUND OF THE INVENTION

The present invention relates to stethoscopes, and particularly to chest pieces therefor, and to methods of utilizing such stethoscopes for monitoring the physiological condition of a patient (human or animal).

Stethoscopes are medical instruments used in listening to sounds produced within the body, particularly in the heart and lungs, for monitoring the physiological condition of a patient. They typically include diaphragm detectors for picking-up sounds of lower frequency, and bell-shaped detectors for picking-up sounds of higher frequency. The earlier stethoscopes transmitted the sounds directly to the physician's ears via flexible rubber tubes; whereas more modern electronic stethoscopes include electrical transducers for converting the sounds to electrical signals and a sound transducer for converting the electrical signals to sounds.

A drawback of electronic stethoscopes having diaphragm-type chest pieces is that a significant part of the original sound is lost because only the central part of the diaphragm is displaced (vibrated) by the sounds, as the outer periphery of the diaphragm is rigidly secured to the rim and metal body of the chest piece.

Another drawback of electronic stethoscopes is that they are highly sensitive to extraneous noises since the noise signals are amplified with the sound signals. Although such stethoscopes commonly include AGC (Automatic Gain Control) circuitry to reduce noise, most of the stethoscopes of this type do not satisfactorily reduce the noise to bearable levels, which is probably the main reason why electronic stethoscopes of this type are not widely used.

In addition, the physician, nurse or other healthcare giver generally examines the subject by moving the chest piece to different ausculation regions of the subject's body while listening to the various sounds picked-up by the chest piece and fed to the headphones worn by the healthcare giver. At times it may be desirable to permit a number of persons to hear the picked-up sounds, for example, for training purposes or for obtaining different opinions as to the meaning of the sounds. The persons may be located at the actual examination site or at a location remote from the examination site. However, because of the high sensitivity of electronic stethoscopes to noise generated by airborne sounds, and particularly because of feedback-resonance problems involved when amplifying signals applied to loudspeakers, electronic stethoscopes have generally not been used with loudspeakers.

### OBJECTS AND BRIEF SUMMARY OF THE INVENTION

One object of the present invention is to provide a stethoscope headpiece capable of utilizing substantially all of the patient's body sounds for generating the electrical signal to be converted to the sounds heard by the user (e.g., physician or other health care giver). Another object is to provide a stethoscope which is capable of being used in environments generating high levels of extraneous noise.

A further object is to provide a stethoscope which is capable of being used with loudspeakers at the examination site and which is capable of producing an electrical signal at the examination site and transmitting the electrical signal to a remote location for reproduction at the remote location. A further object is to provide stethoscope apparatus which is particularly attractive for children to use and which facilitates communication between the healthcare giver and the child. A still further object is to provide a method of monitoring a physiological condition of a subject by using such a chest piece and stethoscope.

According to one aspect of the invention, there is provided a stethoscope chest piece, comprising: a housing having a rigid contact surface to be pressed against a patient's chest for picking-up sounds therefrom; a mass yieldingly mounted with respect to the housing and the contact surface; and an electrical transducer in the housing and cooperable with the mass and the contact surface to generate an electrical signal corresponding to the displacement of the contact surface relative to the mass.

As will be described more particularly below, the sounds picked up by the chest piece in this apparatus are not airborne (acoustical compressional) waves, but rather are body-borne compressional waves picked up by the contact surface of the electrical transducer when displaced relative to the mass. Such compressional waves are much less sensitive to extraneous noise, and therefore may be amplified to the desired intensity without feed back resonance problems.

Such an arrangement is therefore capable of utilizing substantially all the sound-producing displacements (vibrations) of the detector by the patient's body sounds for generating the electrical signals to be converted to the sounds heard by the user, as compared to the conventional stethoscope chest piece including the diaphragm-type detector. The foregoing features enable the apparatus to be used with a loudspeaker, and/or with a communication line, such as a telephone line, connected to a remotely-located unit for reproduction or analysis.

According to another aspect of the invention, there is provided a stethoscope comprising a chest piece including an electrical transducer to be pressed against a patient's chest for picking up sounds therefrom and for producing an electrical signal corresponding to the sounds; processing circuitry for processing the generated electrical signal; and an earpiece including a sound transducer for converting the generated electrical signal to sound; the processing circuitry including a noise detector for detecting extraneous noise above a predetermined threshold, and a disabling circuit controlled by the noise detector for disabling the conversion of the generated electrical signal to sound.

Preferably, the disabling circuit is controlled by the noise detector to disable the conversion of the generated electrical signal to sound for a predetermined interval upon each detection of extraneous noise above the predetermined threshold. The noise detector may restart each predetermined time interval upon each detection of extraneous noise above the predetermined threshold. Also, the processing circuitry may further include a delay circuit for delaying the conversion of the generated electrical signal to sound for a short time interval such that the noise detector disables the conversion of a generated signal to sound also for a short interval immediately before the noise reaches the predetermined threshold.

As will be described more particularly below, such apparatus not only enables a number of persons at various locations to simultaneously listen to the sounds picked up by the chest piece (e.g., for training purposes or for purposes of obtaining additional opinions), but also produces a relatively noise-free signal for all the listeners.

According to another aspect of the invention, the chest piece in the stethoscopic apparatus has the configuration of an animal or other popular figure attractive to children to encourage children to use the apparatus. The apparatus also comprises a reference device resembling a person's chest and having distinctive markings of different ausculation regions commonly used for stethoscopic examination of the body. Preferably, the distinctive markings are of different animals, shapes, popular figures, or numbers. The reference device may be an actual shirt to be worn by the subject, or a picture of such a shirt.

The latter features of the invention make the apparatus particularly attractive to children. They also enable children, and persons in general, to manipulate the chest piece on the subject's body in order to pick up the body sounds and to transmit such body sounds to others in the immediate vicinity or at remote locations (e.g., via the telephone, internet, etc.). Such features also facilitate communication between a healthcare giver at a remote location and a child, or other person, actually manipulating the chest piece according to the directions by the healthcare giver.

According to a still further aspect of the present invention, there is provided a method of monitoring a physiological condition of a patient, comprising: pressing against the patient s chest a chest piece having a housing, a rigid contact surface rigidly connected to the housing, and a mass yieldingly connected to the housing and rigid contact surface; and converting displacements of the rigid contact surface with respect to the mass to sounds indicating the physiological condition of the patient.

Further features and advantages of the invention will be apparent from the description below.

### BRIEF DESCRIPTION OF THE DRAWINGS

The invention is herein described, by way of example only, with reference to the accompanying drawings, wherein:

FIG. 1 illustrates one form of electronic stethoscope constructed in accordance with the present invention;

FIG. 2 is a diagrammatical sectional view illustrating the chest piece in the stethoscope of FIG. 1;

FIG. 3 is a diagrammatical sectional view illustrating a piezoelectric-type chest piece that may be included in the stethoscope of FIG. 1;

FIG. 3a is a sectional view along line a——a of FIG. 3;

FIG. 3b is a large fragmentary view illustrating the piezoelectric device included in the chest piece of FIG. 3;

FIG. 4 is a diagrammatical sectional view illustrating another piezoelectric type chest piece constructed in accordance with the present invention;

FIG. 5 is a diagrammatical sectional view illustrating an electromagnetic-type chest piece constructed in accordance with the present invention;

FIG. 6 is a diagrammatical sectional view illustrating a Hall-effect type chest piece constructed in accordance with the present invention;

FIGS. 7 and 8 are diagrammatical sectional views illustrating two capacitance-type chest pieces constructed in accordance with the present invention;

FIG. 9 is a block diagram diagrammatically illustrating the electrical circuit in the stethoscope of FIG. 1;

FIG. 10 illustrates another form of electronic stethoscope apparatus constructed in accordance with the present invention;

FIG. 11 is a block diagram diagrammatically illustrating the electrical circuit in the stethoscope of FIG. 10;

FIG. 12 illustrates chest pieces configured according to various animals or popular figures to make the chest piece attractive to children;

FIG. 13 is a front view of a shirt (or a picture of a shirt) to be worn by the individual and marked with the ausculation regions for placement of the chest piece; and

FIG. 14 is a rear view of the shirt (or picture) of FIG. 13.

### DESCRIPTION OF PREFERRED EMBODIMENTS

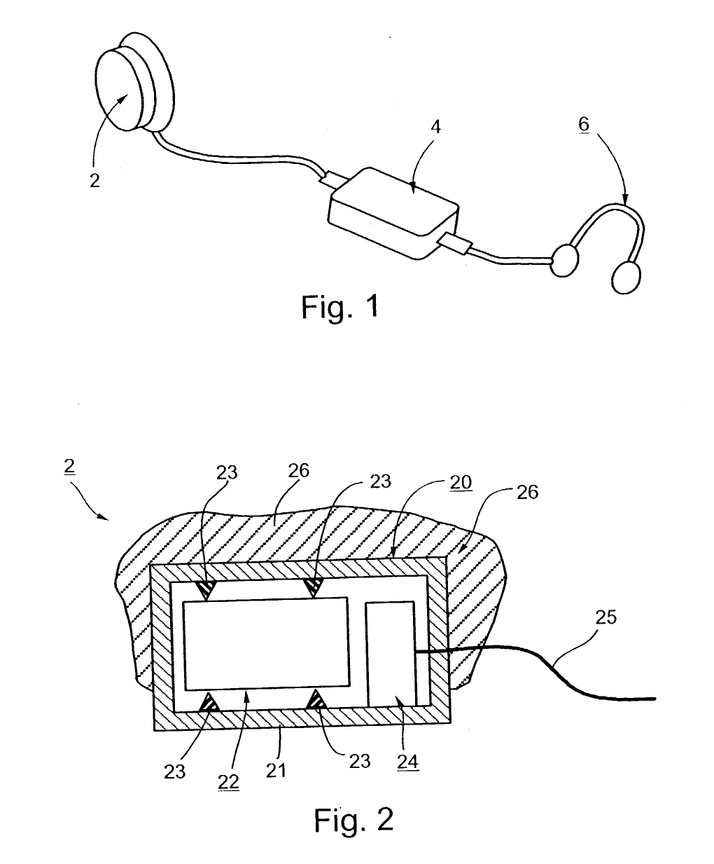

FIG. 1 illustrates one form of electronic stethoscope constructed in accordance with the present invention. It includes: a chest piece, generally designated 2; a circuit box, generally designated 4; and a pair of headphones, generally designated 6. The chest piece 2 is to be pressed against the patient's chest for picking up sounds therefrom and for converting those sounds to an electrical signal; the circuit box 4 amplifies, filters and otherwise processes the electrical signal; and the headphones 6 receive the electrical signal and convert it to sounds which are utilized by the physician for monitoring the physiological condition of the patient.

FIG. 2 diagrammatically illustrates the construction of the chest piece 2 of FIG. 1 according to one aspect of the present invention. Briefly, it includes: a housing 20 of a rigid construction and having a rigid contact surface 21 to be pressed against the patient's chest for picking up the sound signals; a mass, generally designated 22, yieldingly mounted with respect to housing 20 and the contact surface 21 by yielding mounting elements 23; and an electrical transducer, diagrammatically illustrated at 24, located within the housing and cooperable with the contact surface 21 and the mass 22 to generate an electrical signal, outputted via output line 25, corresponding to the displacement of the contact surface 21 relative to the mass 22. Chest piece 2 further includes a handgrip 26 of resilient material on the opposite side of the housing from the rigid contact surface 21 to facilitate pressing the rigid contact surface 21 against the patient's chest.

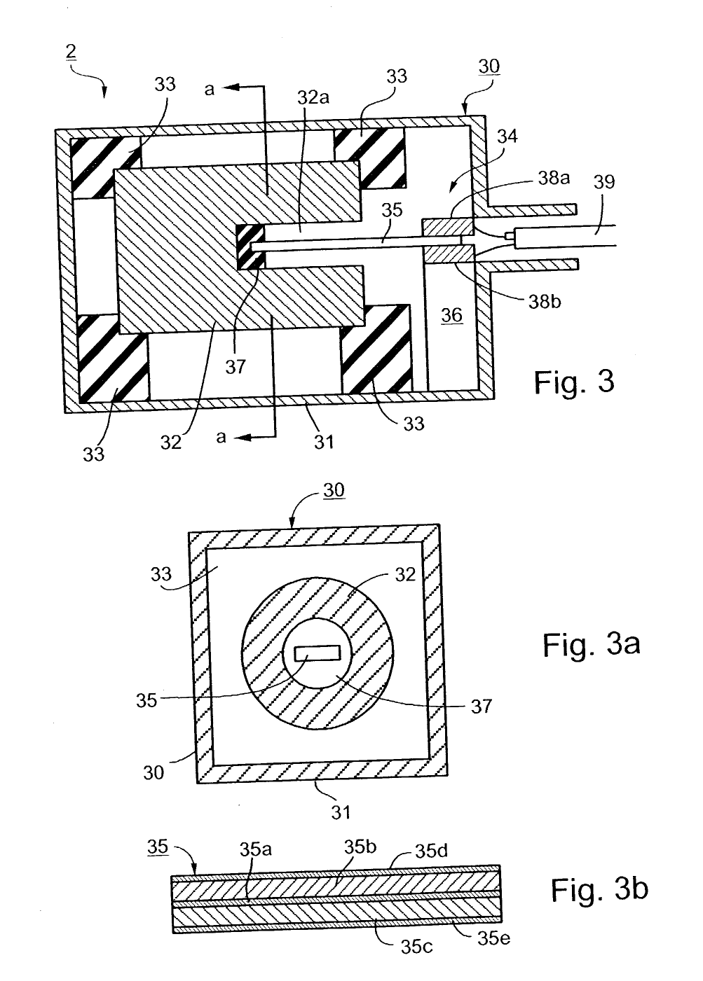

FIGS. 3-8 diagrammatically illustrate various constructions that may be used for the chest piece 2 illustrated in FIG. 2.

FIG. 3 illustrates a construction based on utilizing a piezoelectric transducer in the chest piece. In the illustrated construction, the housing 30 includes a rigid contact surface 31 and a mass 32 yieldingly mounted with respect to the housing and the contact surface by yieldable elements 33. In this case, the mass 32, as shown particularly in FIG. 3a, is a cylindrical body formed with a central bore 32a on one side; and the electrical transducer, generally designated 34, includes a piezoelectric arm 35 rigidly mounted at one end by a post 36 fixed to the rigid contact surface 31 of the housing so as to be displaced with that contact surface when pressed against the patient's chest. The opposite end of piezoelectric arm 35 is mounted within bore 32a of the mass 32 by a cap 37 of resilient material, e.g., rubber. The opposite faces of the piezoelectric arm 35 are engaged by electrical contacts 38a, 38b, to output the generated electrical signal via the output line 39.

As shown in FIGS. 3 and 3a, the yielding mountings of the mass 32 may be in the form of annular resilient rings 33, or annular arrays of resilient elements, yieldingly mounting the opposite ends of the mass with respect to housing 30 and its rigid contact surface 31.

As shown particularly in FIG. 3b, the piezoelectric arm 35 may be of a known bimorph construction including a spring metal leaf 35a having a layer of piezoelectric material 35b, 35c on its opposite faces each layer being covered by an electrical film 35d, 35e engagable by the electrical contacts-38a 38b.

It will be seen that when the contact surface 31 is pressed against the patient's chest the displacements of the chest will be detected by the contact surface and transmitted via post 36 to one end of the piezoelectric arm 35. However, since the opposite end of the piezoelectric arm is mechanically coupled to the mass 32 by resilient cap 37, and since that mass is yieldingly mounted by the resilient elements 33 with respect to housing 30 and its contact surface 31, the displacements of the contact surface 31 will be converted by post 36 to bending movements applied to the respective end of the piezoelectric arm 35, such that the piezoelectric layers 35b, 35c on its opposite faces will generate a voltage proportional to the bending movements. The generated voltage is outputted by contacts 38a, 38b to the output conductor 39.

For purposes of example, the housing 30 may be about 30 mm 30 mm if of rectangular cross-section or about 30 mm in diameter if of circular cross-section; the mass 32 may be about 20g in weight; and the output conductor 39 may be a shielded coaxial cable, with contact 38a connected to its inner conductor and contact 38b connected to its outer shield.

Preferably, cap 37 has some resiliency to prevent breakage of the respective end of the piezoelectric arm 35 and is located at about the center of inertia of the mass 32 along an axis perpendicular to the plane of the contact surface 31. Since contact surface 31 is rigid with the remainder of housing 30, the opposite side of the housing could also be the contact surface.

The mass of the housing 20 should be as low as possible to reduce high frequency attenuation of the skin vibrations. The yielding mass 32 has less effect on this attenuation because of the resilient coupling to the housing provided by the resilient rings 33, particularly at high frequencies.

The relatively large yielding mass 33 improves low frequency sensitivity. The resilient coupling provided by resilient cap 37 reduces some of the sensitivity, but on the other hand makes the structure more robust and reduces its fragility, one of the big disadvantages of accelerometers in general.

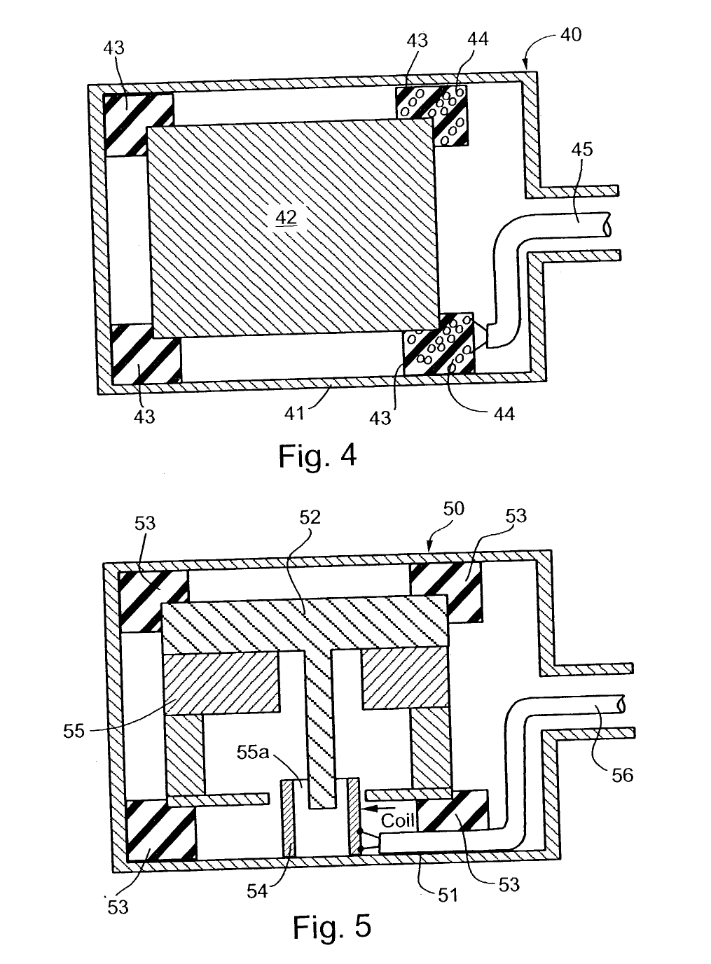

FIG. 4 illustrates another construction, also including a housing 40, a rigid contact surface 41, and a mass 42 yieldingly mounted with respect to the housing and contact surface by yielding elements 43. In this case, however, the electrical transducer for converting the displacements of the contact surface 41 with respect to the mass 42 is in the form of piezoelectric material included in one or more of the yielding mounting elements, as shown at 44, so as to generate the electrical signal corresponding to the displacement of the contact surface 41, and to output such displacement via the output conductor 45.

FIG. 5 also illustrates a construction for the chest piece including a housing 50 having a rigid contact surface 51 and a mass 52 yieldingly mounted by resilient elements 53 with respect to the housing and its contact surface. In this case, however, the displacements of the contact surface 51 are converted to electrical signal by an electromagnet-type transducer.

For this purpose, a coil 54 is rigidly mounted to the contact surface 51. In addition, the mass 52 is made of a magnetic material and carries a magnet 55 to produce a magnetic circuit linking the coil 54 via an air gap 55a.

Thus, as shown in FIG. 5, the coil 54 is movable, by the displacements of the contact surface 51, within the air gap 55a produced by the magnet 55 to generate an electrical signal corresponding to such movements, which electrical signal is outputted via conductor 56.

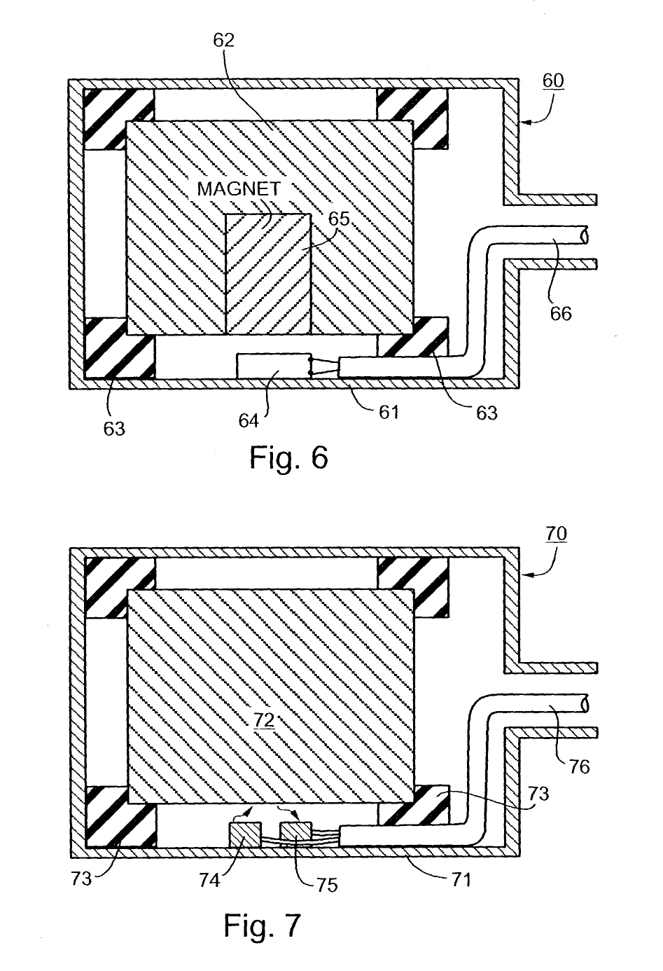

FIG. 6 illustrates another possible construction, based on and including a Hall-effect transducer, for the chest piece 2 illustrated in FIGS. 1 and 2. The FIG. 6 construction also includes a housing 60 having a rigid contact surface 61 and a mass 62 yieldingly mounted by resilient elements 63 to the housing and to the contact surface. In this case, the electrical transducer is a Hall effect device 64 rigidly fixed to the rigid contact surface 61 and movable thereby with respect to a magnet 65 carried by the mass 62, such as to generate a voltage corresponding to the displacements of the Hall effect device 64 with respect to the magnet 65, which voltage is outputted via conductor 66.

FIG. 7 illustrates a chest piece construction including a capacitance-type transducer for generating the outputted electrical signal. Thus, the chest piece also includes a housing 70, a rigid contact surface 71, and a mass 72 yieldingly mounted with respect to the housing 70 and its contact surface 71 by resilient elements 73. In this case, however, the transducer includes a pair of electrodes 74, 75, rigidly mounted in side-by-side relationship to the rigid contact surface 71 so as to be displaceable therewith. Mass 72 is either metal, or includes a metal surface facing the two electrodes 74, 75, such that the movement of the two electrodes with respect to the mass generates an electrical signal, by the capacitance effect corresponding to the displacements of the rigid contact surface 71, and outputted via conductor 76.

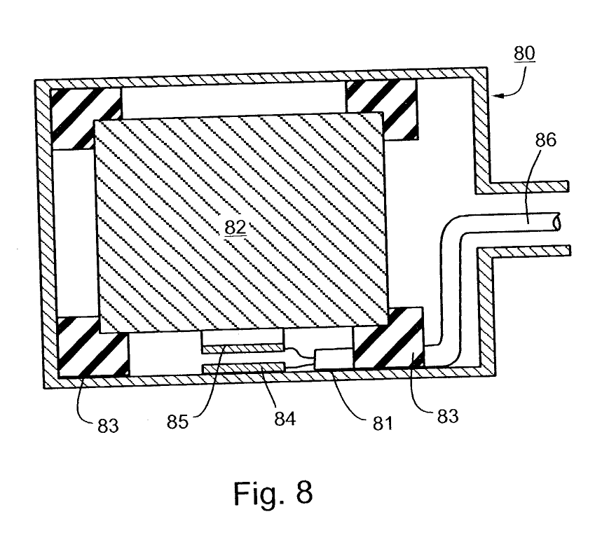

FIG. 8 illustrates a similar construction, also including a housing 80, rigid contact surface 81, and mass 82 yieldingly mounted by resilient elements 83 with respect to the housing and the contact surface. In this case, however, the transducer includes an electrode 84 rigidly mounted to contact surface 81, and another electrode 85 rigidly mounted to the mass 82, to generate, by the capacitance effect, the output signal on conductor 86 corresponding to the displacements of the rigid contact surface 81 with respect to the mass.

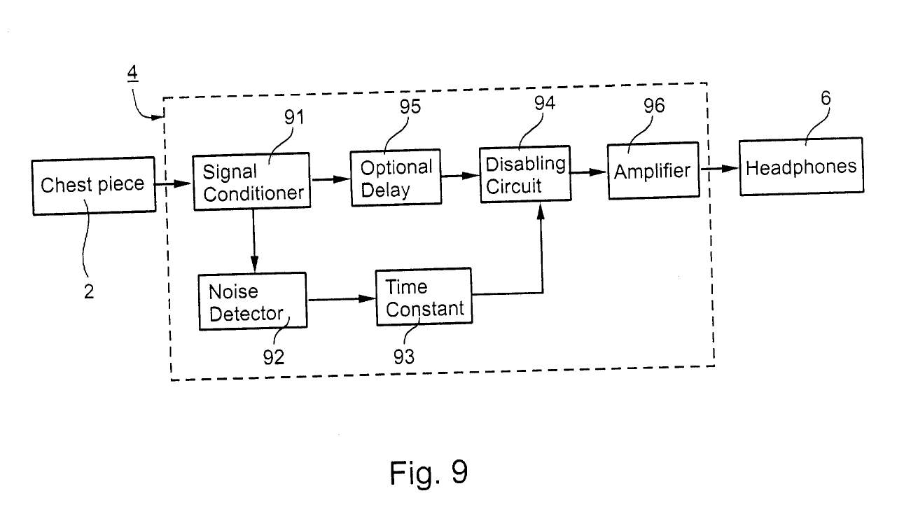

FIG. 9 illustrates a preferred processing circuit which may be used in the circuit box 4 of FIG. 1 connected between the chest piece 2 and the headphones 6 for processing the output signal from the chest piece to minimize external noise before the signal is inputted into the headphones 6.

Thus, the processing circuit 4 includes a signal conditioner 91 which filters and otherwise conditions the electric signal, and a noise detector 92 which detects extraneous noise above a predetermined threshold. If such noise is detected, it triggers a time constant circuit 93 which measures a predetermined time, e.g., one second, and actuates a disabling circuit 94 for disabling the signal from the signal conditioner 91 for the respective time. This disabling may be effected by switching-off the signal from the signal conditioner 91, or by attenuating that signal, before the signal is passed through the amplifier 96 for amplification and then to the headphones 6 for reproduction in the form of sound.

Preferably, the predetermined delay introduced by the time constant circuit 93 is re-triggered each time noise is detected by the detector circuit 92 above the predetermined threshold.

FIG. 9 further illustrates the provision of an optional delay circuit 95 between the signal conditioner circuit 91 and the disabling circuit 94. The delay circuit 95 may be any type of delay device, such as an analog delay device, an SAW (surface acoustic wave) device, or a digital device (e.g., CCD). It delays the conversion of the generated electrical signal to sound for a predetermined time interval, e.g., 1-50 ms. Its purpose is to enable the noise detector 92, which disables the conversion of the generated electrical signal to sound upon the detection of external noise above a predetermined threshold, also to remove from the reproduced sound signal the noise immediately preceding the detected threshold level, thereby producing a cleaner, noise-free sound signal. Such a small delay will generally not even be noticeable.

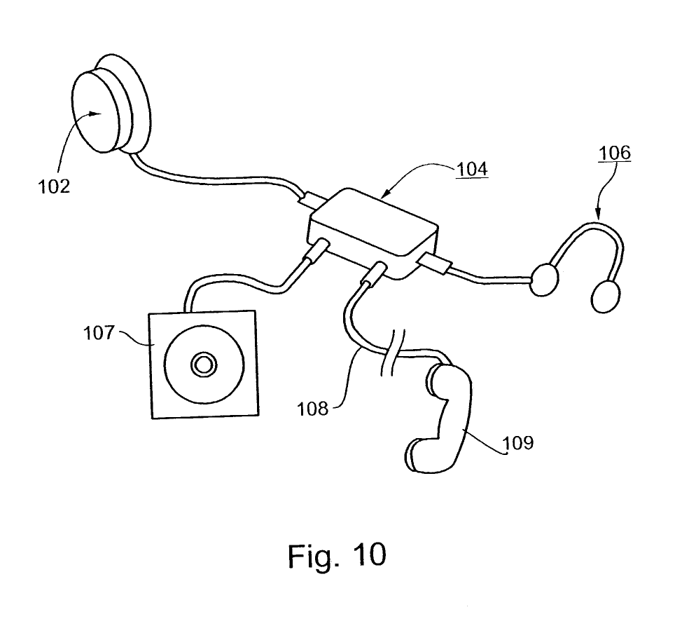

FIG. 10 illustrates another form of electronic stethoscope apparatus constructed in accordance with the present invention. It includes: a chest piece 102; a processing circuit 104; and a pair of headphones 106, corresponding to members 2, 4 and 6, respectively, in FIG. 1.

The illustrated stethoscope apparatus further includes a loudspeaker 107 connected to the processing circuit 104, and an outside communication line 108 also connected to the processing circuit. The loudspeaker 107 reproduces the processed and amplified electrical signal generated by the transducer in the chest piece 102 such that the signal can be clearly heard by a number of people in the immediate vicinity of the subject. Communication line 108 may be a telephone line or the like for transmitting the generated electrical signal to a remote location, such as a telephone unit 109, to enable persons remotely located with respect to the examination site also to hear the sounds generated from the chest piece 102 for purposes of training such persons, of obtaining additional opinions with respect to the meaning of the sounds, etc.

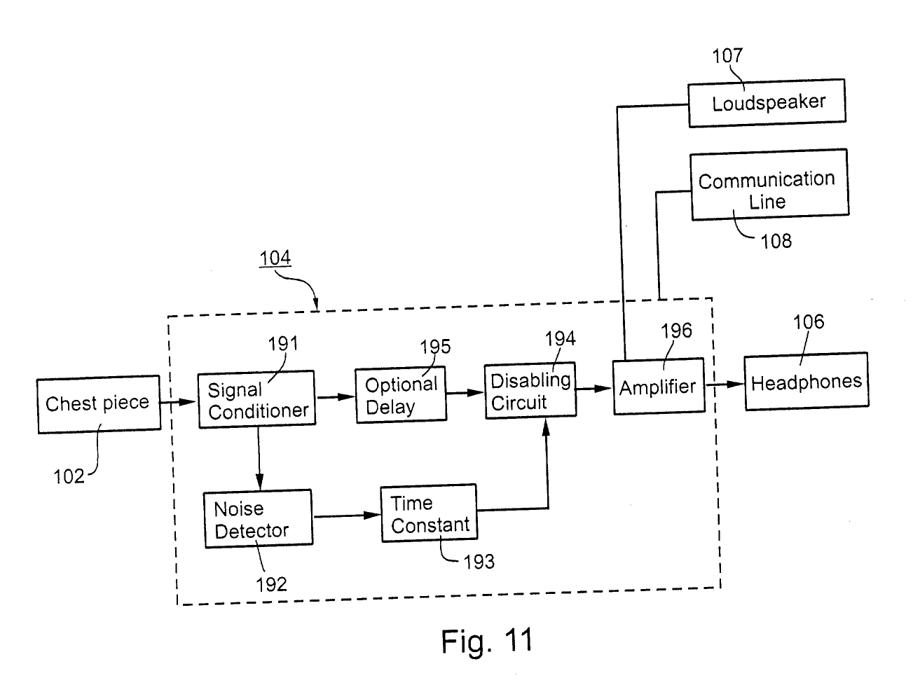

FIG. 11 illustrates a preferred processing circuitry which may be used in the processing circuit 104 of FIG. 10 for processing the output signal from the chest piece to minimize external noise before the signal is inputted into the headphones 106, the loudspeaker 107, and the communication line 108.

Thus, the processing circuit 104 includes a signal conditioner 191, a noise detector 192, a time constant circuit 193, a disabling circuit 194, an optional delay 195, and an amplifier 196, corresponding to elements 91, 92, 93, 94, 95 and 96 respectively, in FIG. 9. As shown in FIG. 10, the amplified signal is fed not only to the headphones 106, but also to loudspeaker 107 and the communication line 108 for reproduction and/or for analysis.

One of the main disadvantages of using accelerometers as signal pickup devices is the mechanical noise from the connection cable. To eliminate this source of noise, the chest piece 102 may be coupled to the processing circuit 104 (or 4 in FIG. 2) by a wireless (e.g., RF) link. For example, the Bluetooth standard may be used for this purpose because of its small dimensions, enabling the chest piece to be linked by a wireless link to other devices, such as a PDA (personal digital assistant), a computer, a mobile phone, etc.

In addition, it is possible to add a microphone to monitor coughs, snoring sounds, and the like. This information may thus be collected while the subject is sleeping, and could be of importance to the physician in diagnosing the physiological condition of the subject. A vibration sensor may also be added to enable monitoring movements of the subject, e.g., for evaluating the quality of sleep by the subject. The vibration sensor may be applied to detect vibrations of the mattress, the bed-frame, or the like.

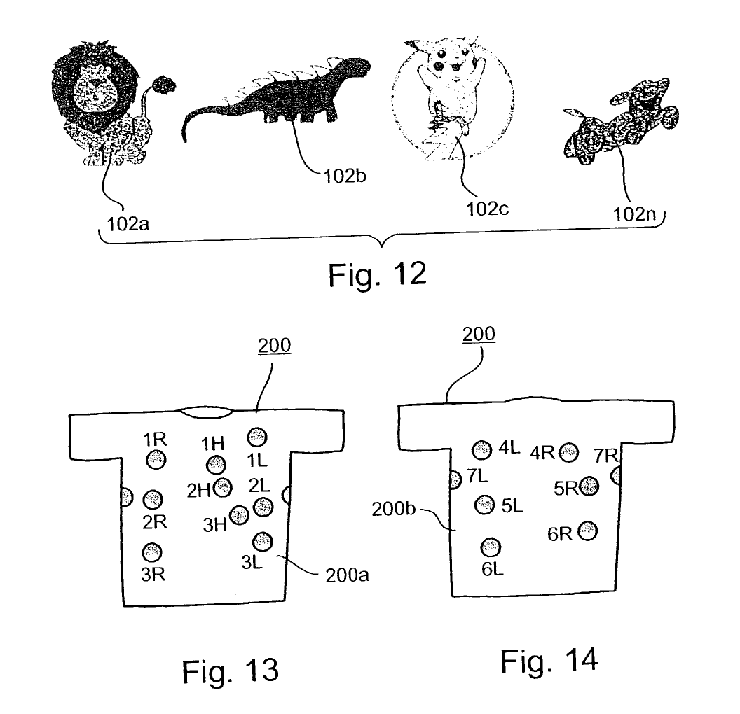

In order to make the use of the stethoscope attractive to children, the chest piece 102 may take the form of an animal or other popular character. FIG. 12 illustrate examples of various types of animals, as shown at 102a - - - 102n, that can be used for the configuration of the chest piece 102.

The apparatus may also be provided with a reference device resembling a persons chest and having distinctive markings of different ausculation regions commonly used for stethoscope examination of the body, which distinctive markings may also be of different animals, popular figures, or numbers. For example, the referenced device may be an actual shirt to be worn by the subject, or a picture of such a shirt.

FIGS. 13 and 14 are front and rear views, respectively, of an actual shirt 200 that may be worn by the subject and marked with the different ausculation regions commonly used for stethoscopic examination. For purposes of example, the front side 200a of the shirt 200 includes three markings 1R, 2R, 3R to indicate three right regions, three markings 1L, 2L, 3L to indicate three left regions; and three markings 1H, 2H, 3H to indicate three heart regions. The rear side 200b of the shirt includes four markings 4L, 5L, 6L, 7L to indicate four left regions; and four markings 4R, 5R, 6R, 7R to indicate four right regions. Thus, a child or other person can be directed to manipulate the chest piece to different ausculation points, and to enable one or more other persons, either in the immediate vicinity of the examination or at a remote location, to hear the actual sounds picked up by the chest piece in a clear and relatively noise-free manner.

While the invention has been described with respect to several preferred embodiments, it will be appreciated that these are set forth merely for purposes of example, and that many other variations and applications of the invention may be made. For example, the chest piece may also be incorporated in a mattress for monitoring heart beats or respirations of a baby or other persons, to thereby serve as an apnea monitor. It may also be used for listening through walls, pipes and the like. Many other variations and applications of the invention will be apparent.

What is claimed is:

1. A stethoscope chest piece, comprising:
a housing having a rigid contact surface to be pressed against a patient's chest for picking-up sounds therefrom;
a mass yieldingly mounted with respect to said housing and said contact surface; and
an electrical transducer in said housing and cooperable with said mass and said contact surface to generate an electrical signal corresponding to the displacement of said contact surface relative to said mass.

2. The chest piece according to claim 1, wherein said electrical transducer includes piezoelectric material to generate said electrical signal by the piezoelectric effect.

3. The chest piece according to claim 2, wherein said electrical transducer further includes an arm having one end rigidly connected to said contact surface so as to be displaced therewith relative to said mass, said piezoelectric material being in the form of a layer on at least one surface of said arm effective to generate said electrical signal corresponding to the displacement of said contact surface relative to said mass.

4. The chest piece according to claim 3, wherein the opposite end of said arm is received within a bore in said mass and is coupled to said mass.

5. The chest piece according to claim 4, wherein said opposite end of the arm is coupled to said mass approximately at the center of inertia of said mass along an axis perpendicular to the plane of said contact surface.

6. The chest piece according to claim 4, wherein said opposite end of the arm is coupled to said mass by a resilient insert received within said bore of the mass and formed with a socket receiving said opposite end of the arm.

7. The chest piece according to claim 3, wherein said arm is of spring metal and includes a layer of piezoelectric material on each of its opposite surfaces.

8. The chest piece according to claim 7, wherein said arm further includes an outer layer of elecrically conductive material on its opposed surfaces for outputting the electrical signal generated by the displacement of said arm by said contact surface with respect to said mass.

9. The chest piece according to claim 2, wherein said piezoelectric material is incorporated in the yielding mountings of the mass with respect to said housing and said contact surface.

10. The chest piece according to claim 1, wherein said mass includes a magnetic body, and said electrical transducer includes a coil magnetically coupled to said magnetic body to generate an electrical signal corresponding to the displacement of said coil relative to said magnetic body.

11. The chest piece according to claim 10, wherein said magnetic body includes an air gap facing said contact surface, and said coil of the electrical transducer is fixed to said contact surface and is movable within said air gap to generate said electrical signal.

12. The chest piece according to claim 1, wherein said mass includes a magnetic body facing said contact surface, and said electrical transducer includes a Hall-effect device fixed to said contact surface and spaced from said magnetic body to generate said electrical signal by the Hall effect corresponding to the displacement of said Hall-effect device relative to said magnetic body.

13. The chest piece according to claim 1, wherein said mass includes a first electrode facing said contact surface, and said electrical transducer includes a second electrode fixed to said contact surface and movable therewith towards and away from said first electrode to generate said electrical signal by capacitance changes between the first and second electrodes.

14. The chest piece according to claim 1, wherein said mass includes a first electrode facing said contact surface, and said electrical transducer includes second and third electrodes fixed to said contact surface in side-by-side relation to each other and movable therewith towards and away from said first electrode to generate said electrical signal by capacitance changes between said electrodes.

15. The chest piece according to claim 1, wherein said mass is yieldingly mounted within said housing with respect to said contact surface by an annular array of resilient elements at each of the opposite ends of said mass.

16. The chest piece according to claim 1, wherein said housing includes a handgrip of resilient material on the opposite side with respect to said rigid contact surface to facilitate pressing the rigid contact surface against a patient's chest.

17. Stethoscope apparatus including a chest piece according to claim 1, processing circuitry for processing said generated electrical signal; and an earpiece including a sound transducer for converting said generated electrical signal to sound.

18. The stethoscope according to claim 17, wherein said processing circuitry includes: a noise detector for detecting extraneous noise above a predetermined threshold; and a disabling circuit controlled by said noise detector for disabling the conversion of the generated electrical signal to sound.

19. The stethoscope according to claim 18, wherein said disabling circuit is controlled by said noise detector to disable the conversion of a generated electrical signal to sound for a predetermined interval upon each detection of extraneous noise above the predetermined threshold.

20. The stethoscope according to claim 19, wherein said noise detector restarts said predetermined time interval upon each detection of extraneous noise above the predetermined threshold.

21. The stethoscope according to claim 19, wherein said processing circuitry further includes a delay circuit for delaying the conversion of the generated electrical signal to sound for a short time interval such that the noise detector will disable the conversion of a generated signal to sound also for the short interval immediately before the noise reaches the predetermined threshold.

22. The apparatus according to claim 17, wherein said apparatus further includes a loudspeaker connected to said processing circuitry for converting the generated electrical signal to sounds.

23. The apparatus according to claim 17, wherein the apparatus further comprises a communication line connected to said processing circuitry for also feeding the generated electrical signal to a remotely-located unit for reproduction or analysis.

24. The apparatus according to claim 23, wherein said communication line is a telephone line transmitting said generated electrical signal to a remotely-located unit for reproduction or analysis.

25. The apparatus according to claim 17, wherein said chest piece has the configuration of a popular figure attractive to children to encourage children to use the stethoscope apparatus.

26. The apparatus according to claim 17, wherein said apparatus further comprises a reference device resembling a person's chest and having distinctive markings of different ausculation regions commonly used for stethoscopic examination of the body.

27. The apparatus according to claim 26, wherein said distinctive markings of different ausculation regions are of different animals, shapes, popular figures, or numbers.

28. The apparatus according to claim 26, wherein said reference device is an actual shirt to be worn by the subject.

29. The apparatus according to claim 26, wherein said reference device is a picture of an actual shirt.

30. A stethoscope, comprising:
a chest piece including an electrical transducer to be pressed against a patient's chest for picking up sounds therefrom and for producing an electrical signal corresponding to the said sounds;
processing circuitry for processing said generated electrical signal; and
an earpiece including a sound transducer for converting said generated electrical signal to sound;
said processing circuitry including a noise detector for detecting extraneous noise above a predetermined threshold, and a disabling circuit controlled by said noise detector for disabling the conversion of the generated electrical signal to sound.

31. The stethoscope according to claim 30, wherein said disabling circuit is controlled by said noise detector to disable the conversion of a generated electrical signal to sound for a predetermined interval upon each detection of extraneous noise above the predetermined threshold.

32. The stethoscope according to claim 31, wherein said noise detector restarts said predetermined time interval upon each detection of extraneous noise above the predetermined threshold.

33. The stethoscope according to claim 31, wherein said processing circuitry further includes a delay circuit for delaying the conversion of the generated electrical signal to sound for a short time interval such that the noise detector will disable the conversion of a generated signal to sound also for the short interval immediately before the noise reaches the predetermined threshold.

34. Stethoscopic apparatus, comprising a chest piece to be pressed against a subject's chest at different ausculation regions thereon for picking-up sounds therefrom; characterized in that said chest piece has the configuration of a popular figure attractive to children to encourage children to use the stethoscope apparatus.

35. The apparatus according to claim 34, wherein said apparatus further comprises a reference device resembling a person's chest and having distinctive markings indicating different ausculation regions thereon commonly used for stethoscopic examination of the body.

36. The apparatus according to claim 35, wherein said distinctive markings of different ausculation regions are of different animals, shapes popular figures, or numbers.

37. The apparatus according to claim 35, wherein said reference device is an actual shirt to be worn by the subject.

38. The apparatus according to claim 35, wherein said reference device is a picture of an actual shirt.

39. A method of monitoring a physiological condition of a patient, comprising:
pressing against the patient's chest a chest piece having a housing, a rigid contact surface rigidly connected to said housing, and a mass yieldingly connected to said housing and rigid contact surface; and
converting displacements of said rigid contact surface with respect to said mass to sounds indicating the physiological condition of the patient.

40. The method according to claim 39, wherein displacements of said rigid contact surface with respect to said mass are converted to sounds by first converting said displacements to an electrical signal, and then converting said electrical signal to sound.

41. The method according to claim 40, wherein said displacements are converted to an electrical signal by a piezoelectric device carried by said housing.

42. The method according to claim 40, wherein said displacements are converted to an electrical signal by an electromagnetic device carried by said housing.

43. The method according to claim 40, wherein said displacements are converted to an electrical signal by a Hall-effect device carried by said housing.

44. The method according to claim 40, wherein said displacements are converted to an electrical signal by a variable-capacitance device carried by said housing.

45. The method according to claim 40, wherein the conversion of said electrical signal to sounds is disabled during each detection of extraneous noise above a predetermined threshold.

46. The method according to claim 45, wherein the disabling of the conversion of said electrical signals to sounds is effected by switching-off the generated electrical signal.

47. The method according to claim 45, wherein the disabling of the conversion of said electrical signals to sounds is effected by attenuating the electrical signal.

48. The method according to claim 45, wherein the conversion of the electrical signals to sounds is disabled for a predetermined period of time which period is restarted by each detection of extraneous noise above the predetermined threshold.

49. The method according to claim 45, wherein the conversion of the electrical signal to sounds is delayed for a short time interval such that the conversion of the generated signal to sound will be disabled also for the short interval before the extraneous noise reaches the predetermined threshold.
